(19) United States
(12) Patent Application Publication
(10) Pub. No.: US 2012/0003672 A1
(43) Pub. Date: Jan. 5, 2012

(54) IN VITRO-METHOD FOR THE DIAGNOSIS, PROGNOSIS, MONITORING AND THERAPY FOLLOW-UP OF DISORDERS ASSOCIATED WITH THE METABOLIC SYNDROME, A CARDIOVASCULAR DISEASE AND/OR INSULIN RESISTANCE

(75) Inventor: Andreas Bergmann, Berlin (DE)

(73) Assignee: B.R.A.H.M.S GmbH, Hennigsdorf (DE)

(21) Appl. No.: 13/126,646

(22) PCT Filed: Oct. 29, 2009
(86) PCT No.: PCT/EP09/07922

§ 371 Date: Sep. 9, 2011

(30) Foreign Application Priority Data
Oct. 31, 2008 (EP) 08168120.7

(51) Int. Cl. G01N 33/53; C12Q 1/54; G01N 21/76

(52) U.S. Cl. 435/7.92; 436/501; 435/14

(57) ABSTRACT

The present invention relates to an in vitro-method for diagnosis, prognosis and/or monitoring/therapy follow-up of a disorder of the metabolic system and/or cardiovascular system and/or insulin resistance in a subject, comprising the determination of the relative level of one or more cardiovascular markers in a sample of a subject and the use of the determined relative level of said one or more cardiovascular peptides for the diagnosis, prognosis and/or monitoring/therapy follow-up of a disorder of the metabolic system and/or cardiovascular system and/or insulin resistance in said subject.

Subject of the invention is an in vitro-method for the diagnosis, prognosis, monitoring and therapy follow-up of disorders associated with the metabolic syndrome, a cardiovascular disease and/or insulin resistance, comprising determining the relative level of one or more cardiovascular markers as well as uses thereof.

Hypertension frequently accompanies obesity, hyperinsulinism and insulin resistance. This type of hypertension is characterized by sodium retention, an increased intravascular volume and increased cardiac stroke volume and output (Messerli F H, et al. 1981 Obesity and essential hypertension. Hemodynamics, intravascular volume, sodium excretion, and plasma renin activity. Arch Intern Med 141:81-5; Stelfox H T, et al. 2006 Hemodynamic monitoring in obese patients: the impact of body mass index on cardiac output and stroke volume. Crit Care Med 34:1243-6). Atrial natriuretic peptide (ANP) and Brain type natriuretic peptide (BNP) are synthesized in myocardial cells as a response to increased wall stress in relation to heart failure or acute myocardial ischemia as prohormones that are cleaved into ANP and BNP and N-terminal proANP (NT-proANP) as well as N-terminal proBNP (NT-proBNP), respectively (Ruskoaho H, 2003 Cardiac hormones as diagnostic tools in heart failure. Endocr Rev 24:341-56; Potter L R, et al. 2006 Natriuretic peptides, their receptors, and cyclic guanosine monophosphate-dependent signaling functions. Endocr Rev 27:47-72). High levels of natriuretic peptides are new promising cardiovascular (CV) risk markers and have been associated with high blood pressure (BP), left ventricular hypertrophy, and albuminuria (Olsen M H, et al. 2005 N-terminal pro brain natriuretic peptide is inversely related to metabolic cardiovascular risk factors and the metabolic syndrome. Hypertension 46:660-6). Several large studies indeed observed independent associations of mortality and natriuretic peptide levels (Wang T J, et al. 2004 Plasma natriuretic peptide levels and the risk of cardiovascular events and death. N Engl J Med 350:655-63; Bibbins-Domingo K, et al. 2007 N-terminal fragment of the prohormone brain-type natriuretic peptide (NT-proBNP), cardiovascular events, and mortality in patients with stable coronary heart disease. Jama 297:169-76; Kistorp C, et al. 2005 N-terminal pro-brain natriuretic peptide, C-reactive protein, and urinary albumin levels as predictors of mortality and cardiovascular events in older adults. Jama 293:1609-16; von Haehling, et al. 2007 Comparison of midregional pro-atrial natriuretic peptide with N-terminal pro-B-type natriuretic peptide in predicting survival in patients with chronic heart failure. J Am Coll Cardiol 50:1973-80).

Recent studies have described that natriuretic peptide levels are suppressed in obesity (Wang T J, et al. 2004 Impact of obesity on plasma natriuretic peptide levels. Circulation 109:594-600; Das S R, et al. 2005 Impact of body mass and body composition on circulating levels of natriuretic peptides: results from the Dallas Heart Study. Circulation 112:2163-8). Since obesity is associated with salt retention and increased cardiac output it would be expected to produce elevated natriuretic peptide levels. That obesity seemed to have the opposite effect appeared counterintuitive and was attributed to non-hemodynamic factors. Wang and colleagues therefore postulated that this inverse relationship may be due to increased expression of the natriuretic peptide clearance receptor (NPR-C) by adipose tissue resulting in increased clearance of natriuretic peptides in obese subjects (Wang T J, et al. 2004 Impact of obesity on plasma natriuretic peptide levels. Circulation 109:594-600). However, Das and colleagues determined lean and fat mass by DEXA in the Dallas heart study and observed an association of lower BNP levels with lean rather than fat mass (Das S R, et al. 2005 Impact of body mass and body composition on circulating levels of natriuretic peptides: results from the Dallas Heart Study. Circulation 112:2163-8).

Several studies observed an association of natriuretic peptides with further components of the metabolic syndrome (Olsen M H, et al. 2008 Cardiovascular risk prediction by N-terminal pro brain natriuretic peptide and high sensitivity C-reactive protein is affected by age and sex. J Hypertens 26:26-34; Wang T J, et al. 2007 Association of plasma natriuretic peptide levels with metabolic risk factors in ambulatory individuals. Circulation 115:1345-53). Elevated waist circumference, elevated triglycerides, reduced HDL, and elevated fasting glucose (Wang T J, et al. 2007 Association of plasma natriuretic peptide levels with metabolic risk factors in ambulatory individuals. Circulation 115:1345-53) were associated with lower plasma ANP levels, and somewhat less with lower BNP levels in the Framingham heart study. In a Danish study, an association of BNP with BMI, insulin, glucose, triglycerides and hypertension was observed (Olsen M H, et al. 2005 N-terminal pro brain natriuretic peptide is inversely related to metabolic cardiovascular risk factors and the metabolic syndrome. Hypertension 46:660-6). Although these studies demonstrated close links of the natriuretic peptides to several traits of the metabolic syndrome, the mechanisms behind these associations have remained elusive.

Nutritional approaches reducing postprandial insulin levels are highly effective in reducing blood pressure (Appel L J, et al. 2005 Effects of protein, monounsaturated fat, and carbohydrate intake on blood pressure and serum lipids: results of the OmniHeart randomized trial. Jama 294:2455-64) and the postprandial state is believed to play an important role in the early stages of the metabolic syndrome and particularly in the development of atherosclerosis (Hanefeld M, et al. 1999 Postprandial plasma glucose is an independent risk factor for increased carotid intima-media thickness in non-diabetic individuals. Atherosclerosis 144:229-35; Hanefeld M, et al. 2007 The challenge of the Metabolic Syndrome. Horm Metab Res 39:625-6).

Object of the present invention was the diagnosis, prognosis and/or monitoring and/or therapy follow-up of disorders associated with the metabolic syndrome and/or a disease associated with the cardiovascular system and/or insulin resistance in a subject, wherein the relative change of one or more cardiovascular marker is determined in said subject.

It was found that insulin induces the suppression of cardiovascular markers, in particular MR-proANP₅₃₋₉₀ and there was a significant positive correlation between the insulin sensitivity and the relative change of natriuretic peptides, in particular of MR-proANP₅₃₋₉₀. This connection provides a direct link from insulin resistance to hypertension in the metabolic syndrome. The finding of this connection further leads to methods for the diagnosis, prognosis and/or monitoring and/or therapy follow-up of disorders associated with the metabolic syndrome and/or a disease associated with the cardiovascular system and/or insulin resistance in a subject.

Thus, subject of the present invention is an in vitro-method for diagnosis, prognosis and/or monitoring/therapy follow-up of a disorder of the metabolic system and/or cardiovascular system and/or insulin resistance in a subject, comprising:
providing a sample from said subject,
determining the relative level of one or more cardiovascular marker in said sample,
using the relative level of said one or more cardiovascular peptides for the diagnosis, prognosis and/or monitoring/therapy follow-up of a disorder of the metabolic system and/or cardiovascular system and/or insulin resistance in said subject.

In a preferred embodiment of the invention the postprandial relative level of one or more cardiovascular markers is determined.

According to the present invention “relative level” is defined as being the relative to concentration based on a basal value, which can be mathematically expressed as follows:

$$X = 100 \times \frac{[ postprandial ]}{[ basal ]}$$

wherein X is the change of the level of one or more cardiovascular markers in percent and [postprandial] and [basal] are the postprandial and basal level, respectively.

It can also be specified as percentaged change of the concentration relative to the basal value, which can be mathematically expressed as follows:

$$X = 100 - \frac{[ postprandial ]}{[ basal ]} \times 100 or$$

$$X = \frac{[ postprandial ]}{[ basal ]} \times 100 - 100$$

In a preferred embodiment of the inventive method the postprandial relative level of one or more cardiovascular markers is determined

In the context of the present invention the term “postprandial” refers to the period of time after a foodstuff (nutrition) and/or a beverage and/or a medicament is ingested by or applied otherwise to the subject, which may also be in the context of a diet and/or nutrition regimen.

The postprandial level may also be determined in the context of a glucose tolerance or glucose challenge test. For example an oral glucose tolerance test (oGTT) could be carried out (after fasting overnight) by the oral application of 75 g of glucose (usually given as a glucose solution) that should be drunk within 5 minutes. Blood samples are drawn at baseline (before glucose application) and at different time intervals, e.g. 15, 30, 60, 90, 120 or 180 minutes.

Glucose tolerance can also be determined with the intravenous glucose tolerance test (ivGTT). After an overnight fast a glucose dose adjusted for body weight (e.g. 0.3 g/kg to a maximum of e.g. 25g) is administered by a catheter e.g. in the forearm. Blood samples are drawn at baseline (before the application of glucose) and at different time intervals, e.g. 1, 2, 3, 5, 7, 10, 15, 20, 30, 45, 60, 90, 120 or 240 minutes.

In addition, the postprandial level may also be determined in the context of a hyperinsulinemic, euglycemic clamp test to investigate and quantify insulin resistance. This test measures the amount of glucose necessary to compensate for an increased insulin level without causing hypoglycaemia. Through a peripheral vein, insulin is infused at 10-120 mU per m² per minute. In order to compensate for the insulin infusion, glucose 20% is infused to maintain blood sugar levels between 5 and 5.5 mmol/1. The rate of glucose infusion is determined by checking the blood sugar levels every 5 to 10 minutes. The rate of glucose infusion during the last 30 minutes of the test determines insulin sensitivity. If high levels (7.5 mg/min or higher) are required, the patient is insulin-sensitive. Very low levels (4.0 mg/min or lower) indicate that the body is resistant to insulin action. Levels between 4.0 and 7.5 mg/min are not definitive and suggest “impaired glucose tolerance,” an early sign of insulin resistance.

In the context of the present invention cardiovascular marker means a peptide and/or protein providing diagnosis and/or prognosis and/or monitoring of disorders associated with the metabolic syndrome (e.g. myocardial infarction, coronary artery disease, heart failure, type II diabetes, obesity, hypertension) selected from the group of natriuretic peptides (e.g. atrial natriuretic peptide, brain natriuretic peptide), adrenomedullin, endothelins, vasopressin. The basal level of the cardiovascular markers depends on such factors as the subject's age, body mass index, genetic predisposition for certain conditions/family history, gender and ethnic background of the patient, as well as on the overall health status of said subject. The present invention, however, is based on the finding that in contrast to this, the relative change from the basal level of the cardiovascular markers to the postprandial level of the cardiovascular markers is essentially independent from these factors and strongly depends on foodstuff and/or beverage and/or diet and/or nutrition regimen and/or medicament which is administered to the subject.

The cardiovascular marker is preferably selected from the group comprising ANP, BNP, ET-1, ADM, AVP and fragments thereof and pro-hormones and fragments thereof.

In an especially preferred embodiment of the inventive method the relative level of one or more cardiovascular markers is determined with an assay having a lower detection limit of 1 nmol/L or lower, preferably 100 pmol/L or lower, more preferably 10 pmol/L or lower, even more preferably 1 pmol/L or lower, most preferably 0.5 pmol/L or lower. Furthermore, the assay preferably has an interassay precision of <30%, more preferably, <20% in the normal range. Furthermore, the assay preferably has an intraassay precision of <10%, more preferably, <5% in the measuring range. Hereby “intraassay precision” specifies the deviance between measurements within a single batch of a specific assay, and “interassay precision” specifies the deviance between measurements within multiple batches of a specific assay, which may be carried out in different locations, on different days, by different operators. Thus, the aforementioned terms are related to a measure of the reproducibility of results obtained with the concerned assays. “Measuring range” specifies the upper and lower limit of detection of an assay.

The assay is at least sensitive enough to detect changes and variances as increase and as decrease. For a healthy subject the normal range of a given biomarker corresponds to a Gaussian distribution.

An embodiment of the invention is further an in vitro-method according to the present invention, further comprising

a) determining the basal level of one or more cardiovascular markers in said subject

b) determining the postprandial level of said one or more cardiovascular markers.

c) calculating the relative level of one or more cardiovascular markers from the values obtained in steps a and b.

Hereby, the ingestion, intake or other form of application of said foodstuff and/or beverage and/or diet and/or nutrition regimen and/or medicament is correlated to its influence on the level of said one or more cardiovascular markers in said patient in terms of a relative decrease of said level.

In the context of the present invention, the term “basal level” refers to the individual level of a certain compound, molecule or metabolite, such as a cardiovascular peptide, which a subject has without the influence of factors such as a foodstuff, a beverage, a diet, a nutrition regimen or a medicament. Said basal level is individually determined for each subject after approximately 12 hours of fasting. Fasting hereby means that the subject does not ingest or otherwise consume foodstuffs, beverages or medicaments for a certain amount of time, except water and/or indispensable medication.

As mentioned herein, an “assay” or “diagnostic assay” can be of any type applied in the field of diagnostics. Such an assay may be based on the binding of an analyte to be detected to one or more capture probes with a certain affinity. Concerning the interaction between capture molecules and target molecules or molecules of interest, the affinity constant is preferably greater than 10⁸ M⁻¹.

As mentioned herein in the context of pro-hormones and other peptides, the term “fragment” refers to smaller proteins or peptides derivable from larger proteins or peptides, which hence comprise a partial sequence of the larger protein or peptide. Said fragments are derivable from the larger proteins or peptides by saponification of one or more of its peptide bonds.

“Fragments” of the cardiovascular markers proANP, proBNP, proET-1, proADM and pro-AVP preferably relate to fragments of at least 6 amino acids in length, most preferably at least 12 amino acid residues in length. Such fragments are preferably detectable with immunological assays as described herein.

In the context of the present invention, “capture molecules” are molecules which may be used to bind target molecules or molecules of interest, i.e. analytes (i.e. in the context of the present invention the cardiovascular peptide(s)), from a sample. Capture molecules must thus be shaped adequately, both spatially and in terms of surface features, such as surface charge, hydrophobicity, hydrophilicity, presence or absence of lewis donors and/or acceptors, to specifically bind the target molecules or molecules of interest. Hereby, the binding may for instance be mediated by ionic, van-der-Waals, pi-pi, sigma-pi, hydrophobic or hydrogen bond interactions or a combination of two or more of the aforementioned interactions between the capture molecules and the target molecules or molecules of interest. In the context of the present invention, capture molecules may for instance be selected from the group comprising a nucleic acid molecule, a carbohydrate molecule, a RNA molecule, a protein, an antibody, a peptide or a glycoprotein. Preferably, the capture molecules are antibodies, including fragments thereof with sufficient affinity to a target or molecule of interest, and including recombinant antibodies or recombinant antibody fragments, as well as chemically and/or biochemically modified derivatives of said antibodies or fragments derived from the variant chain with a length of at least 12 amino acids thereof.

The preferred detection methods comprise immunoassays in various formats such as for instance radioimmunoassay (RIA), chemiluminescence- and fluorescence-immunoassays, Enzyme-linked immunoassays (ELISA), Luminex-based bead arrays, protein microarray assays, and rapid test formats such as for instance immunochromatographic strip tests.

The assays can be homogenous or heterogeneous assays, competitive and non-competitive sandwich assays. In a particularly preferred embodiment, the assay is in the form of a sandwich assay, which is a non-competitive immunoassay, wherein the molecule to be detected and/or quantified is bound to a first antibody and to a second antibody. The first antibody may be bound to a solid phase, e.g. a bead, a surface of a well or other container, a chip or a strip, and the second antibody is an antibody which is labeled, e.g. with a dye, with a radioisotope, or a reactive or catalytically active moiety. The amount of labeled antibody bound to the analyte is then measured by an appropriate method. The general composition and procedures involved with “sandwich assays” are well-established and known to the skilled person (The Immunoassay Handbook, Ed. David Wild, Elsevier LTD, Oxford; 3rd ed. (May 2005), ISBN-13: 978-0080445267; Hultschig C et al., Curr Opin Chem Biol. 2006 February; 10(1):4-10. PMID: 16376134, incorporated herein by reference).

In a particularly preferred embodiment the assay comprises two capture molecules, preferably antibodies which are both present as dispersions in a liquid reaction mixture, wherein a first labelling component is attached to the first capture molecule, wherein said first labelling component is part of a labelling system based on fluorescence- or chemiluminescence-quenching or amplification, and a second labelling component of said marking system is attached to the second capture molecule, so that upon binding of both capture molecules to the analyte a measurable signal is generated that allows for the detection of the formed sandwich complexes in the solution comprising the sample.

Even more preferred, said labelling system comprises rare earth cryptates or rare earth chelates in combination with a fluorescence dye or chemiluminescence dye, in particular a dye of the cyanine type.

In the context of the present invention, fluorescence based assays comprise the use of dyes, which may for instance be selected from the group comprising FAM (5- or 6-carboxyfluorescein), VIC, NED, Fluorescein, Fluoresceinisothiocyanate (FITC), IRD-700/800, Cyanine dyes, such as CY3, CY5, CY3.5, CY5.5, Cy7, Xanthen, 6-Carboxy-2′,4′,7′,4,7-hexachlorofluorescein (HEX), TET, 6-Carboxy-4′,5′-dichloro-2′,7′-dimethodyfluorescein (JOE), N,N,N′,N′-Tetramethyl-6-carboxyrhodamine (TAMRA), 6-Carboxy-X-rhodamine (ROX), 5-Carboxyrhodamine-6G (R6G5), 6-carboxyrhodamine-6G (RG6), Rhodamine, Rhodamine Green, Rhodamine Red, Rhodamine 110, BODIPY dyes, such as BODIPY TMR, Oregon Green, Coumarines such as Umbelliferone, Benzimides, such as Hoechst 33258; Phenanthridines, such as Texas Red, Yakima Yellow, Alexa Fluor, PET, Ethidiumbromide, Acridinium dyes, Carbazol dyes, Phenoxazine dyes, Porphyrin dyes, Polymethin dyes, and the like.

In the context of the present invention, chemiluminescence based assays comprise the use of dyes, based on the physical principles described for chemiluminescent materials in Kirk-Othmer, Encyclopedia of chemical technology, 4^{th} ed., executive editor, J. I. Kroschwitz; editor, M. Howe-Grant, John Wiley & Sons, 1993, vol. 15, p. 518-562, incorporated herein by reference, including citations on pages 551-562. Preferred chemiluminescent dyes are acridiniumesters.

In another preferred embodiment the basal level of said cardiovascular marker and the post-prandial level of said cardiovascular marker in said patient are determined with an immunoassay.

As outlined above, the diagnostic assay can be of any type applied in the field of diagnostics, including but not restricted to assay methods based on enzymatic reactions, luminescence, fluorescence, or radiochemicals. The preferred detection methods comprise strip tests, radioimmunoassay, chemiluminescence- and fluorescence-immunoassay, Immunoblot assay, Enzyme-linked immunoassay (ELISA), Luminex-based bead arrays, and protein microarray assay. The assay types can further be microtiter plate-based, chip-based, bead-based, wherein the biomarker proteins can be attached to the surface or are in solution. The assays can be homogenous or heterogeneous assays, sandwich assays, competitive and non-competive assays (The Immunoassay Handbook, Ed. David Wild, Elsevier LTD, Oxford; 3rd ed. (May 2005), ISBN-13: 978-0080445267; Hultschig C et al., Curr Opin Chem Biol. 2006 February; 10(1):4-10. PMID: 16376134).

In the most preferred embodiment of the invention an immunoassay is used as described in (Morgenthaler N G et al; 2004 Clin Chem 50:234-6).

In an especially preferred embodiment of the in vitro-method according to the invention one of the cardiovascular markers is proANP. It is even more preferred that one of the markers is midregional proANP. Mostly preferred is midregional proANP₅₃₋₉₀. MR-proANP₅₃₋₉₀ specifies the midregional pro-atrial natriuretic peptide, which comprises amino acids 53 to 90 of the pro-atrial natriuretic peptide (proANP).

AVP in the context of the present invention relates to arginine vasopressin (=vasopressin) or fragments thereof or precursors or fragments thereof. A preferred fragment of a precursor of AVP is C-terminal proAVP (CT-proAVP or Copeptin). CT-proAVP₁₀₇₋₁₄₅ (or CT-pre-proAVP₁₂₆₋₁₆₄) is a particularly preferred cardiovascular marker in the context of the present invention.

ADM in the context of the present invention relates to adrenomedullin or fragments thereof or precursors or fragments thereof. A preferred fragment of a precursor of ADM is mid-regional proADM (MR-proADM). MR-proADM₂₄₋₇₁ (or MR-preproADM₄₅₋₉₂) is a particularly preferred cardiovascular marker in the context of the present invention.

ET-1 in the context of the present invention relates to endothelin 1 or fragments thereof or precursors or fragments thereof. A preferred fragment of a precursor of ET-1 is C-terminal-proET1 (CT-proET1). CT-proET-1₁₅₁₋₁₉₅ (or CT-preproET-1₁₆₈₋₂₁₂) is a particularly preferred cardiovascular marker in the context of the present invention.

BNP in the context of the present invention relates to brain natriuretic peptide or fragments thereof or precursors or fragments thereof. A preferred fragment of a precursor of BNP is N-terminal proBNP (NT-proBNP). NT-proBNP is a particularly preferred cardiovascular marker in the context of the present invention.

In a preferred embodiment of the invention one or more of the cardiovascular markers is selected from the group comprising proANP or fragments thereof (preferably MR-proANP, more preferably MR-proANP₅₃₋₉₉), pro-BNP or fragments thereof (preferably NT-proBNP), pro-ET-1 or fragments thereof (preferably CT-proET1, more preferably CT-proET-1₁₅₁₋₁₉₅), pro-AVP or fragments thereof (preferably copeptin, more preferably CT-proAVP107-145), pro-ADM or fragments thereof (preferably MR-proADM, more preferably MR-proADM₂₄₋₇₁).

According to the present invention it is preferred that at least one of the markers is a peptide selected from the group comprising natriurectic peptides, endothelin-1, vasopressin, adrenomedullin, as well as propeptides thereof and fragments of at least 3 amino acids thereof, preferably more than 5, more preferably more than 6, even more preferably more than 7, even more preferably more than 10, even more preferably more than 12, even more preferably more than 15, most preferably 20 or more.

In a very preferred embodiment of the invention at least one of the cardiovascular markers is atrial natriurectic peptide (ANP) or a propeptide and fragments of at least 3 amino acids thereof, preferably more than 5, more preferably more than 6, most preferably more than 7.

In an especially preferred embodiment at least one of the cardiovascular markers is MR-proANP₅₃₋₉₉ or fragments of at least 3 amino acids thereof preferably more than 5, more preferably more than 6, most preferably more than 7.

The invention also relates to particular embodiments of the in vitro-method according the invention, wherein additionally the level of one or more further markers or clinical parameters having predictive value for classifying the propensity of said patient for a disorder of the metabolic system and/or cardiovascular system is determined, wherein the clinical parameters may be any parameter which might influence said propensity, such as for instance age, gender, prior history of diseases, in particular hypertension, obesity, in particular central obesity, body mass index, genetic predisposition/family history, ethnic background, patient's habits which affect said propensity, such as smoking, alcohol consumption, diet, exercise or medication.

In another preferred embodiment of the in vitro-method according to the invention the post-prandial level of said one or more cardiovascular markers is determined within 4 hours, preferably within 2 hours, more preferably between 15 and 60 minutes after administration of said foodstuff and/or beverage and/or diet and/or nutrition regimen.

In another preferred embodiment of the in vitro-method according to the invention the post-prandial influence of said foodstuff and/or beverage and/or diet and/or nutrition regimen on the relative level of one or more cardiovascular peptides is monitored over a prolonged period, preferably over a period of one week, more preferably one month, even more preferably two months, even more preferably half a year, even more preferably during the entire duration of the disease. For a chronic disease, the monitoring may be performed for the whole lifetime of a patient.

The invention may also involve comparing the relative level of a cardiovascular marker for the individual with a predetermined value. The predetermined value can take a variety of forms. It can be single cut-off value, such as for instance a median or mean or the 75th, 90th, 95th or 99th percentile of a population. It can be established based upon comparative groups, such as where the risk in one defined group is double the risk in another defined group. It can be a range, for example, where the tested population is divided equally (or unequally) into groups, such as a low-risk group, a medium-risk group and a high-risk group, or into quartiles, the lowest quartile being individuals with the lowest risk and the highest quartile being individuals with the highest risk.

The predetermined value can vary among particular populations selected, depending on their habits, ethnicity, genetics etc. For example, an apparently healthy, non-smoker population (no detectable disease, particularly no diabetes mellitus) might have a different ‘normal’ range of markers than a smoking population or a population the members of which have a disease of the cardiovascular system and/or the metabolic system. Accordingly, the predetermined values selected may take into account the category in which an individual falls. Appropriate ranges and categories can be selected with no more than routine experimentation by those of ordinary skill in the art.

Threshold levels can be obtained for instance from a Kaplan-Meier analysis, where the occurrence of a disease is correlated with the quartiles of the cardiovascular markers in the population. According to this analysis, subjects with cardiovascular marker levels above the 75th percentile have a significantly increased risk for getting the diseases according to the invention. This result is further supported by Cox regression analysis with full adjustment for classical risk factors: The highest quartile versus all other subjects is highly significantly associated with increased risk for getting a disease according to the invention.

Other preferred cut-off values are for instance the 90th, 95th or 99th percentile of a normal population. By using a higher percentile than the 75th percentile, one reduces the number of false positive subjects identified, but one might miss to identify subjects, who are at moderate, albeit still increased risk. Thus, one might adopt the cut-off value depending on whether it is considered more appropriate to identify most of the subjects at risk at the expense of also identifying “false positives”, or whether it is considered more appropriate to identify mainly the subjects at high risk at the expense of missing several subjects at moderate risk.

Other mathematical possibilities to calculate an individual's risk by using the individual's cardiovascular marker level value and other prognostic laboratory and clinical parameters are for instance the NRI (Net Reclassification Index) or the IDI (Integrated Discrimination Index). The indices can be calculated according to Pencina (Pencina M J, et al.: Evaluating the added predictive ability of a new marker: from area under the ROC curve to reclassification and beyond. Stat Med. 2008; 27:157-172).

In a particular embodiment of the invention, a postprandial relative decrease of the level of said one or more cardiovascular markers of more than 5%, preferably more than 10%, more preferably more than 15%, even more preferably more than 20% in said subject is correlated to the diagnosis and a negative prognosis of a disorder of the metabolic system and/or cardiovascular system and/or insulin resistance and to an increased risk for the subject for contracting a disorder of the metabolic system and/or cardiovascular system and/or insulin resistance.

In another particular embodiment of the invention, a postprandial relative increase of the level of said one or more cardiovascular markers of more than 5%, preferably more than 10%, more preferably more than 15%, even more preferably more than 20% in said subject is correlated to a positive prognosis of a disorder of the metabolic system and/or cardiovascular system and/or insulin resistance and to an increased risk for the subject for contracting a disorder of the metabolic system and/or cardiovascular system and/or insulin resistance.

Subject of the invention is further the use of an assay, preferably having a sensitivity of 1 nmol/L or lower, preferably 100 pmol/L or lower, more preferably 10 pmol/L or lower, even more preferably 1 pmol/L or lower, most preferably 0.5 pmol/L or lower for determining the change of the level of one or more cardiovascular markers of a subject relative to the basal level of said markers of said subject, wherein the assay is capable of detecting a decrease.

It is important to note that the capability of the assay used in the present invention to measure a decrease in the level of said one or more cardiovascular markers is critical, wherein the decrease leads to very low levels of said one or more cardiovascular markers. Thus, the assay used herein preferably is ultra-sensitive in order to be capable of measuring a decrease in the level of said one or more cardiovascular markers, in subjects in which the basal level lies within the 97.5¹ percentile of said level in the healthy population.

Subject of the invention is further the use of an assay as described above, for determining the postprandial change of the level of one or more cardiovascular markers of a subject relative to the basal level of said markers of said subject.

It is preferred that the assay is an immunoassay.

Subject of the invention is further the use of an assay according as outlined above, wherein said change in the level of one or more cardiovascular markers is used to diagnose, predict and/or monitor a subject comprising a disorder associated with the metabolic syndrome.

In another embodiment of the invention said subject is a human being having a medical condition associated with the metabolic syndrome.

In a particular embodiment said change in the level of one or more cardiovascular markers is used for the diagnosis/prognosis and/or monitoring of diabetes, in particular type 2 diabetes.

As used herein, the term “metabolic syndrome” refers to the aggregation of several risk factors for cardiovascular diseases and type II diabetes, as defined by the American Heart Association (AHA) and National Heart, Lung and Blood Institute (NHLBI) (Grundy et al. 2005. Circulation 112: 2735-3752), incorporated herein by reference Important components of the metabolic syndrome are, among others, glucose intolerance and dyslipidemias, hypertension and central obesity. The metabolic syndrome is diagnosed if 3 of the following 5 criteria are fulfilled: elevated waist circumference (≧102 cm in male and ≧88 cm in female), elevated triglycerides (≧150 mg/dL/1.7 mmol/L, respectively, or drug treatment for elevated triglycerides), reduced HDL-cholesterol (<40 mg/dL/1.03 mmol/L, respectively, in male; <50 mg/dL/1.3 mmol/L, respectively, in female; or drug treatment for reduced HDL-cholesterol), elevated blood pressure 130 mm Hg systolic blood pressure or ≧85 mm Hg diastolic blood pressure or on antihypertensive drug treatment in a patient with a history of hypertension) and elevated fasting glucose (≧100 mg/dL or drug treatment for elevated glucose).

A more recent definition with some modifications has been given by the International Diabetes Federation (http://www.idf.org/webdata/docs/IDF_Meta_def_final.pdf).

In another preferred embodiment of the invention, the condition associated with the metabolic syndrome is selected from the group comprising myocardial infarction (MI), coronary syndromes, congestive heart failure (CHF), coronary artery disease (atherosclerosis), stroke, transient ischemic attacks (TIA), periphery artery disease, cardiomyopathy, diabetes mellitus type II, renal failure and/or patients with one or more symptoms of the above mentioned diseases, e.g. obesity, hypertension, headache, chest pain and dyspnea.

Insulin resistance (IR) is a state in which a given concentration of insulin produces a less-than-expected biological effect. Insulin resistance has also been arbitrarily defined as the requirement of 200 or more units of insulin per day to attain glycemic control and to prevent ketosis. High plasma levels of insulin and glucose due to insulin resistance often lead to metabolic syndrome and type II diabetes, including its complications. Symptoms of IR may comprise fatigue, brain fogginess, inability to focus, low blood sugar, intestinal bloating, sleepiness, weight gain, fat storage, difficulty losing weight, increased blood triglyceride levels, increased blood pressure, and depression.

A cardiovascular disease is characterized by the dysfunction of the heart muscle or the blood vessel system supplying the heart, brain and other vital organs. The term “cardiovascular disease” covers a wide array of disorders including arteriosclerosis, coronary artery disease, heart valve disease, arrhythmia, heart failure, hypertension, orthostatic hypotension, shock, endocarditis, diseases of the aorta and its branches, disorders of the peripheral vascular system, congenital heart disease, stroke.

Subject if the present invention is also the use of an assay for determining the change of the level of one or more cardiovascular markers of a subject relative to the basal level of said markers of said subject, wherein the assay is capable of detecting a decrease of the level of said one or more cardiovascular markers and capable of detecting an increase of the level of said one or more cardiovascular markers.

In a preferred embodiment of the assay the change is an increase or a decrease, and wherein the assay has sensitivity of 1 nmol/L or lower.

Another embodiment of the invention is the use of the assay for determining the postprandial change of the level of one or more cardiovascular markers of a subject relative the basal level of said markers of said subject.

The assay is used for diagnosis, prognosis and/or monitoring/therapy follow-up of a disorder of the metabolic system and/or cardiovascular system and/or insulin resistance in a subject.

Subject of the invention is further the use of a cardiovascular peptide for diagnosis, prognosis and/or therapy follow-up of a disorder of the metabolic system and/or cardiovascular system of a patient.

The term “subject” as used herein refers to a living human or non-human organism. Preferably herein the subject is a human subject.

The term “sample” as used herein refers to a sample of bodily fluid obtained for the purpose of diagnosis, prognosis, or evaluation of a subject of interest, such as a patient. Preferred test samples include blood, serum, plasma, cerebrospinal fluid, urine, saliva, sputum, and pleural effusions. In addition, one of skill in the art would realize that some test samples would be more readily analyzed following a fractionation or purification procedure, for example, separation of whole blood into serum or plasma components.

Thus, in a preferred embodiment of the invention the sample is selected from the group comprising a blood sample, a serum sample, a plasma sample, a cerebrospinal fluid sample, a saliva sample and a urine sample or an extract of any of the aforementioned samples. Preferably, the sample is a blood sample, most preferably a serum sample or a plasma sample.

The relative levels of the markers as obtained by the methods or by the use of the assays according to the present invention may be analyzed in a number of fashions well known to a person skilled in the art. For example, each assay result obtained may be compared to a “normal” value, or a value indicating a particular disease or outcome. A particular diagnosis/prognosis may depend upon the comparison of each assay result to such a value, which may be referred to as a diagnostic or prognostic “threshold”.

The sensitivity and specificity of a diagnostic and/or prognostic test depends on more than just the analytical “quality” of the test, they also depend on the definition of what constitutes an abnormal result. In practice, Receiver Operating Characteristic curves (ROC curves), are typically calculated by plotting the value of a variable versus its relative frequency in “normal” (i.e. apparently healthy) and “disease” populations (i.e. patients suffering from diabetes, insulin resistance and/or metabolic syndrome). For any particular marker, a distribution of marker levels for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a condition. ROC curves can be used even when test results don't necessarily give an accurate number. As long as one can rank results, one can create a ROC curve. For example, results of a test on “disease” samples might be ranked according to degree (e.g. 1=low, 2=normal, and 3=high). This ranking can be correlated to results in the “normal” population, and a ROC curve created. These methods are well known in the art. See, e.g., Hanley et al., Radiology 143: 29-36 (1982). Preferably, a threshold is selected to provide a ROC curve area of greater than about 0.5, more preferably greater than about 0.7, still more preferably greater than about 0.8, even more preferably greater than about 0.85, and most preferably greater than about 0.9. The term “about” in this context refers to +/−5% of a given measurement.

The horizontal axis of the ROC curve represents (1-specificity), which increases with the rate of false positives. The vertical axis of the curve represents sensitivity, which increases with the rate of true positives. Thus, for a particular cut-off selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

In certain embodiments, particular thresholds for one or more markers in a panel are not relied upon to determine if a profile of marker levels obtained from a subject are indicative of a particular diagnosis/prognosis. Rather, the present invention may utilize an evaluation of a marker panel “profile” as a unitary whole. A particular “fingerprint” pattern of changes in such a panel of markers may, in effect, act as a specific diagnostic or prognostic indicator.

As discussed herein, that pattern of changes may be obtained from a single sample, or from temporal changes in one or more members of the panel (or a panel response value). A panel herein refers to a set of markers.

As described herein after, a panel response value is preferably determined by plotting ROC curves for the sensitivity of a particular panel of markers versus 1-(specificity) for the panel at various cut-offs. In these methods, a profile of marker measurements from a subject is considered together to provide a global probability (expressed either as a numeric score or as a percentage risk) of a diagnosis or prognosis. In such embodiments, an increase in a certain subset of markers may be sufficient to indicate a particular diagnosis/prognosis in one patient, while an increase in a different subset of markers may be sufficient to indicate the same or a different diagnosis/prognosis in another patient. Weighting factors may also be applied to one or more markers in a panel, for example, when a marker is of particularly high utility in identifying a particular diagnosis/prognosis, it may be weighted so that at a given level it alone is sufficient to signal a positive result. Likewise, a weighting factor may provide that no given level of a particular marker is sufficient to signal a positive result, but only signals a result when another marker also contributes to the analysis.

In certain embodiments, markers and/or marker panels are selected to exhibit at least about 70% sensitivity, more preferably at least about 80% sensitivity, even more preferably at least about 85% sensitivity, still more preferably at least about 90% sensitivity, and most preferably at least about 95% sensitivity, combined with at least about 70% specificity, more preferably at least about 80% specificity, even more preferably at least about 85% specificity, still more preferably at least about 90% specificity, and most preferably at least about 95% specificity. In particularly preferred embodiments, both the sensitivity and specificity are at least about 75%, more preferably at least about 80%, even more preferably at least about 85%, still more preferably at least about 90%, and most preferably at least about 95%. The term “about” in this context refers to +/−5% of a given measurement.

In other embodiments, a positive likelihood ratio, negative likelihood ratio, odds ratio, or hazard ratio is used as a measure of a test's ability to predict risk or diagnose a disease. In the case of a positive likelihood ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In the case of a negative likelihood ratio, a value of 1 indicates that a negative result is equally likely among subjects in both the “diseased” and “control” groups; a value greater than 1 indicates that a negative result is more likely in the test group; and a value less than 1 indicates that a negative result is more likely in the control group. In certain preferred embodiments, markers and/or marker panels are preferably selected to exhibit a positive or negative likelihood ratio of at least about 1.5 or more or about 0.67 or less, more preferably at least about 2 or more or about 0.5 or less, still more preferably at least about 5 or more or about 0.2 or less, even more preferably at to least about 10 or more or about 0.1 or less, and most preferably at least about 20 or more or about 0.05 or less. The term “about” in this context refers to +/−5% of a given measurement.

In the case of an odds ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In certain preferred embodiments, markers and/or marker panels are preferably selected to exhibit an odds ratio of at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less. The term “about” in this context refers to +/−5% of a given measurement.

In the case of a hazard ratio, a value of 1 indicates that the relative risk of an endpoint (e.g., death) is equal in both the “diseased” and “control” groups; a value greater than 1 indicates that the risk is greater in the diseased group; and a value less than 1 indicates that the risk is greater in the control group. In certain preferred embodiments, markers and/or marker panels are preferably selected to exhibit a hazard ratio of at least about 1.1 or more or about 0.91 or less, more preferably at least about 1.25 or more or about 0.8 or less, still more preferably at least about 1.5 or more or about 0.67 or less, even more preferably at least about 2 or more or about 0.5 or less, and most preferably at least about 2.5 or more or about 0.4 or less. The term “about” in this context refers to +/5% of a given measurement.

The skilled artisan will understand that associating a diagnostic or prognostic indicator, with a diagnosis or with a prognostic risk of a future clinical outcome is a statistical analysis. For example, a relative marker level of greater than X may signal that a patient is more likely to suffer from an adverse outcome than patients with a relative level less than or equal to X, as determined by a level of statistical significance. Statistical significance is often determined by comparing two or more populations, and determining a confidence interval and/or a p value. See, e.g., Dowdy and Wearden, Statistics for Research, John Wiley & Sons, New York, 1983. Preferred confidence intervals of the invention are 90%, 95%, 97.5%, 98%, 99%, 99.5%, 99.9% and 99.99%, while preferred p values are 0.1, 0.05, 0.025, 0.02, 0.01, 0.005, 0.001, and 0.0001.

Sequences

The amino acid sequence of the precursor peptide of Adrenomedullin (pre-pro-Adrenomedullin) is given in SEQ ID NO: 1. Pro-Adrenomedullin relates to amino acid residues 22 to 185 of the sequence of pre-pro-Adrenomedullin The amino acid sequence of pro-Adrenomedullin (pro-ADM) is given in SEQ ID NO:2. The pro-ADM N-terminal 20 peptide (PAMP) relates to amino acid residues 22-41 of pre-proADM. The amino acid sequence of PAMP is given in SEQ ID NO:3. MR-pro-Adrenomedullin (MR-pro-ADM) relates to amino acid residues 45-92 of pre-pro-ADM. The amino acid sequence of MR-pro-ADM is provided in SEQ ID NO:4. The amino acid sequence of mature Adrenomedullin (ADM) is given in SEQ ID NO:5.

The amino acid sequence of ANP is given in SEQ ID NO:8. The sequence of the 153 amino acid pre-pro-ANP is shown in SEQ ID NO:6. Upon cleavage of an N-terminal signal peptide (25 amino acids) and the two C-terminal amino acids (127/128) proANP (SEQ ID NO:7) is released. ANP comprises residues 99-126 from the C-terminus of the precursor prohormone pro-ANP. This prohormone is cleaved into the mature 28 amino acid peptide ANP, also known as ANP (1-28) or α-ANP, and the amino terminal fragment ANP (1-98) (NT-proANP, SEQ ID NO:9). Mid-regional proANP (MR-proANP) is defined as NT-proANP or any fragments thereof comprising at least amino acid residues 53-90 (SEQ ID NO:10) of proANP.

The sequence of the 164 amino acid precursor peptide of Vasopressin (pre-pro-Vasopressin) is given in SEQ ID NO:11. Pro-Vasopressin relates to the amino acid residues 29 to 164 of the sequence of pre-pro-Vasopressin. The amino acid sequence of pro-Vasopressin is given in SEQ ID NO:12. Pro-Vasopressin is cleaved into mature Vasopressin, Neurophysin II and C-terminal proVasopressin (CT-proAVP or Copeptin). Vasopressin relates to the amino acid residues 20 to 28 of pre-pro-Vasopressin. The amino acid sequence of Vasopressin is shown in SEQ ID NO:13. Coeptin relates to amino acid residues 126 to 164 of pre-pro-Vasopressin. The amino acid sequence of Copeptin is provided in SEQ ID NO:14. Neurophysin II comprises the amino acid residues 32 to 124 of pre-pro-Vasopressin and its sequence is shown in SEQ ID NO:15.

The sequence of the 212 amino acid precursor peptide of Endothelin-1(pre-pro-Endothelin-1) is given in SEQ ID NO:16. Pro-ET-1 relates to the amino acid residues 18 to 212 of the sequence of pre-pro-ET-1. The amino acid sequence of pro-ET-1 is given in SEQ ID NO:17. Pro-ET-1 is cleaved into mature ET-1, big-ET-1 and C-terminal proET-1 (CT-proET-1). ET-1 relates to the amino acid residues 53 to 73 of pre-pro-ET-1. The amino acid sequence of ET-1 is shown in SEQ ID NO:18. CT-proET-1 relates to amino acid residues 168 to 212 of pre-pro-ET-1. The amino acid sequence of CT-proET-1 is provided in SEQ ID NO:19. Big-ET-1 comprises the amino acid residues 53 to 90 of pre-pro-ET-1 and its sequence is shown in SEQ ID NO:20.

The sequence of the 134 amino acid precursor peptide of brain natriuretic peptide (pre-pro-BNP) is given in SEQ ID NO:21. Pro-BNP relates to amino acid residues 27 to 134 of pro-pro-BNP. The sequence of pro-BNP is shown in SEQ ID NO:22. Pro-BNP is cleaved into N-terminal pro-BNP (NT-pro-BNP) and mature BNP. NT-pro-BNP comprises the amino acid residues 27 to 102 and its sequence is shown in SEQ ID NO:23. The SEQ ID NO:24 shows the sequence of BNP comprising the amino acid residues 103 to 134 of the pre-pro-BNP peptide.

```
SEQ ID NO: 1 (amino acid sequence of pre-pro-ADM):
    1 MKLVSVALMY LGSLAFLGAD TARLDVASEF RKKWNKWALS RGKRELRMSS
  51 SYPTGLADVK AGPAQTLIRP QDMKGASRSP EDSSPDAARI RVKRYRQSMN
101 NFQGLRSFGC RFGTCTVQKL AHQIYQFTDK DKDNVAPRSK ISPQGYGRRR
151 RRSLPEAGPG RTLVSSKPQA HGAPAPPSGS APHFL
SEQ ID NO: 2 (amino acid sequence of pro-ADM):
    1 ARLDVASEFR KKWNKWALSR GKRELRMSSS YPTGLADVKA GPAQTLIRPQ
  51 DMKGASRSPE DSSPDAARIR VKRYRQSMNN FQGLRSFGCR FGTCTVQKLA
101 HQIYQFTDKD KDNVAPRSKI SPQGYGRRRR RSLPEAGPGR TLVSSKPQAH
151 GAPAPPSGSA PHFL
SEQ ID NO: 3 (amino acid sequence of pro-ADM N20):
    1 ARLDVASEFR KKWNKWALSR
SEQ ID NO: 4 (amino acid sequence of MR-pro-ADM):
    1 ELRMSSSYPT GLADVKAGPA QTLIRPQDMK GASRSPEDSS
SEQ ID NO: 5 (amino acid sequence of ADM):
    1 YRQSMNNFQG LRSFGCRFGT CTVQKLAHQI YQFTDKDKDN VAPRSKISPQ
  51 GY
SEQ ID NO: 6 (amino acid sequence of pre-pro-ANP):
    1 MSSFSTTTVS FLLLLAFQLL GQTRANPMYN AVSNADLMDF KNLLDHLEEK
  51 MPLEDEVVPP QVLSEPNEEA GAALSPLPEV PPWTGEVSPA QRDGGALGRG
101 PWDSSDRSAL LKSKLRALLT APRSLRRSSC FGGRMDRIGA QSGLGCNSFR
151 YRR
SEQ ID NO: 7 (amino acid sequence of pro-ANP):
    1 NPMYNAVSNA DLMDFKNLLD HLEEKMPLED EVVPPQVLSE PNEEAGAALS
  51 PLPEVPPWTG EVSPAQRDGG ALGRGPWDSS DRSALLKSKL RALLTAPRSL
101 RRSSCFGGRM DRIGAQSGLG CNSFRY
SEQ ID NO: 8 (amino acid sequence of ANP):
    1 SLRRSSCFGG RMDRIGAQSG LGCNSFRY
SEQ ID NO: 9 (amino acid sequence of NT-proANP):
    1 NPMYNAVSNA DLMDFKNLLD HLEEKMPLED EVVPPQVLSE PNEEAGAALS
  51 PLPEVPPWTG EVSPAQRDGG ALGRGPWDSS DRSALLKSKL RALLTAPR
SEQ ID NO: 10 (amino acid sequence of amino acids 53-90 of
proANP):
    1 PEVPPWTGEV SPAQRDGGAL GRGPWDSSDR SALLKSKL
SEQ ID NO: 11 (amino acid sequence of pre-pro-AVP):
    1 MPDTMLPACF LGLLAFSSAC YFQNCPRGGK RAMSDLELRQ CLPCGPGGKG
  51 RCFGPSICCA DELGCFVGTA EALRCQEENY LPSPCQSGQK ACGSGGRCAA
101 FGVCCNDESC VTEPECREGF HRRARASDRS NATQLDGPAG ALLLRLVQLA
151 GAPEPFEPAQ PDAY
SEQ ID NO: 12 (amino acid sequence of pro-AVP):
    1 CYFQNCPRGG KRAMSDLELR QCLPCGPGGK GRCFGPSICC ADELGCFVGT
  51 AEALRCQEEN YLPSPCQSGQ KACGSGGRCA AFGVCCNDES CVTEPECREG
101 FHRRARASDR SNATQLDGPA GALLLRLVQL AGAPEPFEPA QPDAY
SEQ ID NO: 13 (amino acid sequence of AVP):
    1 CYFQNCPRG
SEQ ID NO: 14 (amino acid sequence of CT-pre-proAVP or
Copeptin):
    1 ASDRSNATQL DGPAGALLLR LVQLAGAPEP FEPAQPDAY
SEQ ID NO: 15 (amino acid sequence of Neurophysin II):
    1 AMSDLELRQC LPCGPGGKGR CFGPSICCAD ELGCFVGTAE ALRCQEENYL
  51 PSPCQSGQKA CGSGGRCAAF GVCCNDESCV TEPECREGFH RRA
SEQ ID NO: 16 (amino acid sequence of pre-pro-ET-1):
    1 MDYLLMIFSL LFVACQGAPE TAVLGAELSA VGENGGEKPT PSPPWRLRRS
  51 KRCSCSSLMD KECVYFCHLD IIWVNTPEHV VPYGLGSPRS KRALENLLPT
101 KATDRENRCQ CASQKDKKCW NFCQAGKELR AEDIMEKDWN NHKKGKDCSK
151 LGKKCIYQQL VRGRKIRRSS EEHLRQTRSE TMRNSVKSSF HDPKLKGKPS
201 RERYVTHNRA HW
SEQ ID NO: 17 (amino acid sequence of pro-ET-1):
    1 APETAVLGAE LSAVGENGGE KPTPSPPWRL RRSKRCSCSS LMDKECVYFC
  51 HLDIIWVNTP EHVVPYGLGS PRSKRALENL LPTKATDREN RCQCASQKDK
101 KCWNFCQAGK ELRAEDIMEK DWNNHKKGKD CSKLGKKCIY QQLVRGRKIR
151 RSSEEHLRQT RSETMRNSVK SSFHDPKLKG KPSRERYVTH NRAHW
SEQ ID NO: 18 (amino acid sequence of ET-1):
    1 CSCSSLMDKE CVYFCHLDII W
SEQ ID NO: 19 (amino acid sequence of CT-pro-ET-1):
    1 RSSEEHLRQT RSETMRNSVK SSFHDPKLKG KPSRERYVTH NRAHW
SEQ ID NO: 20 (amino acid sequence of Big-ET-1):
    1 CSCSSLMDKE CVYFCHLDII WVNTPEHVVP YGLGSPRS
SEQ ID NO: 21 (amino acid sequence of pre-pro-BNP):
    1 MDPQTAPSRA LLLLLFLHLA FLGGRSHPLG SPGSASDLET SGLQEQRNHL
  51 QGKLSELQVE QTSLEPLQES PRPTGVWKSR EVATEGIRGH RKMVLYTLRA
101 PRSPKMVQGS GCFGRKMDRI SSSSGLGCKV LRRH
SEQ ID NO: 22 (amino acid sequence of pro-BNP):
    1 HPLGSPGSAS DLETSGLQEQ RNHLQGKLSE LQVEQTSLEP LQESPRPTGV
  51 WKSREVATEG IRGHRKMVLY TLRAPRSPKM VQGSGCFGRK MDRISSSSGL
101 GCKVLRRH
SEQ ID NO: 23 (amino acid sequence of NT-pro-BNP):
    1 HPLGSPGSAS DLETSGLQEQ RNHLQGKLSE LQVEQTSLEP LQESPRPTGV
  51 WKSREVATEG IRGHRKMVLY TLRAPR
SEQ ID NO: 24 (amino acid sequence of BNP):
    1 SPKMVQGSGC FGRKMDRISS SSGLGCKVLR RH
```

### DESCRIPTION OF DRAWINGS

FIG. 1

sequence of pre-pro-ADM

FIG. 2

sequence of pro-ADM

FIG. 3

sequence of pro-ADM N20

FIG. 4

sequence of MR-pro-ADM

FIG. 5

sequence of ADM

FIG. 6

sequence of pre-pro-ANP

FIG. 7

sequence of pro-ANP

FIG. 8

sequence of ANP

FIG. 9

sequence of NT-proANP

FIG. 10

sequence of amino acids 53-90 of proANP

FIG. 11

sequence of pre-pro-AVP

FIG. 12

sequence of pro-AVP

FIG. 13

sequence of AVP

FIG. 14

sequence of CT-pre-proAVP or Copeptin

FIG. 15

sequence of Neurophysin II

FIG. 16

sequence of pre-pro-ET-1

FIG. 17

sequence of pro-ET-1

FIG. 18

sequence of ET-1

FIG. 19

sequence of CT-pro-ET-1

FIG. 20

sequence of Big-ET-1

FIG. 21

sequence of pre-pro-BNP

FIG. 22

sequence of pro-BNP

FIG. 23

sequence of NT-pro-BNP

FIG. 24

sequence of BNP

FIG. 25

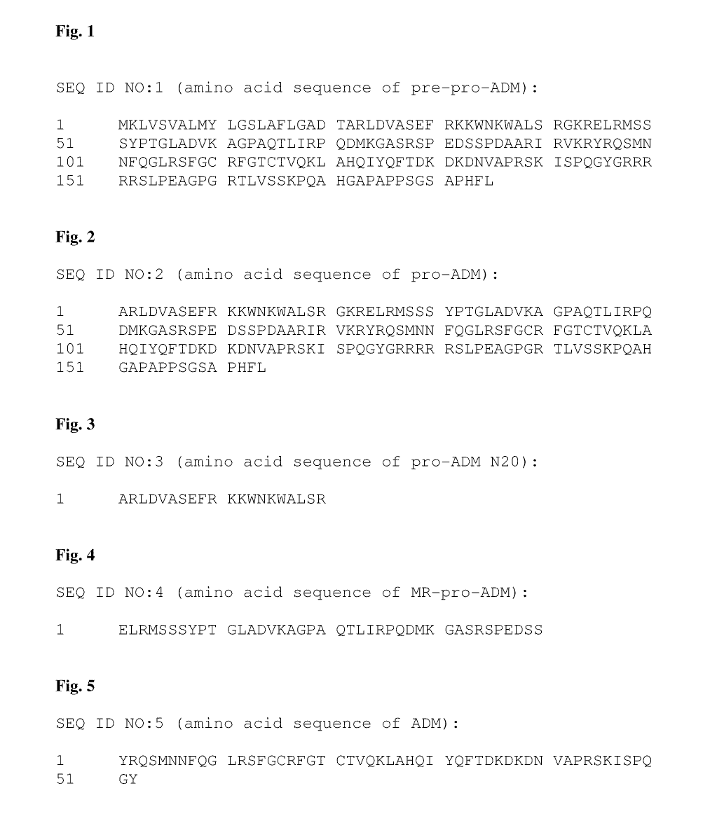

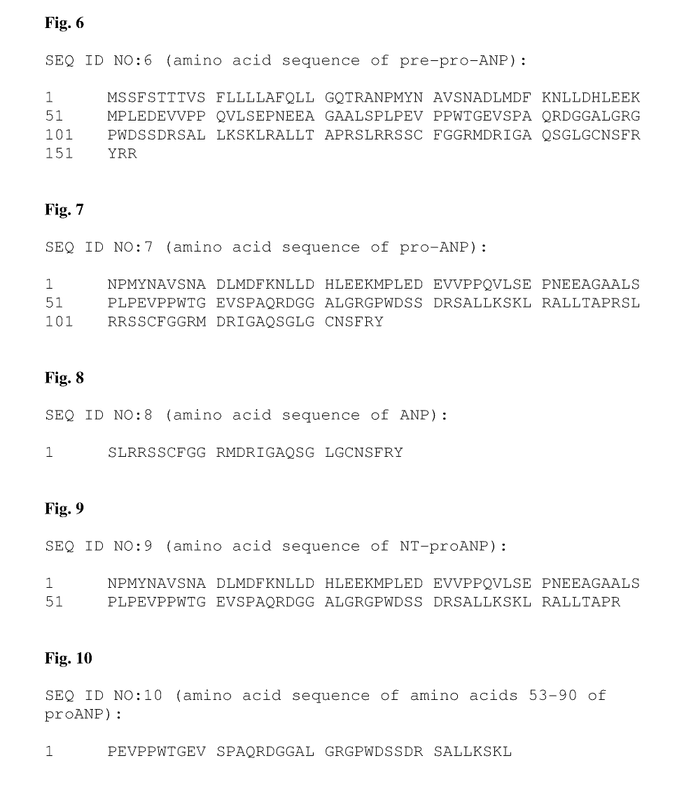

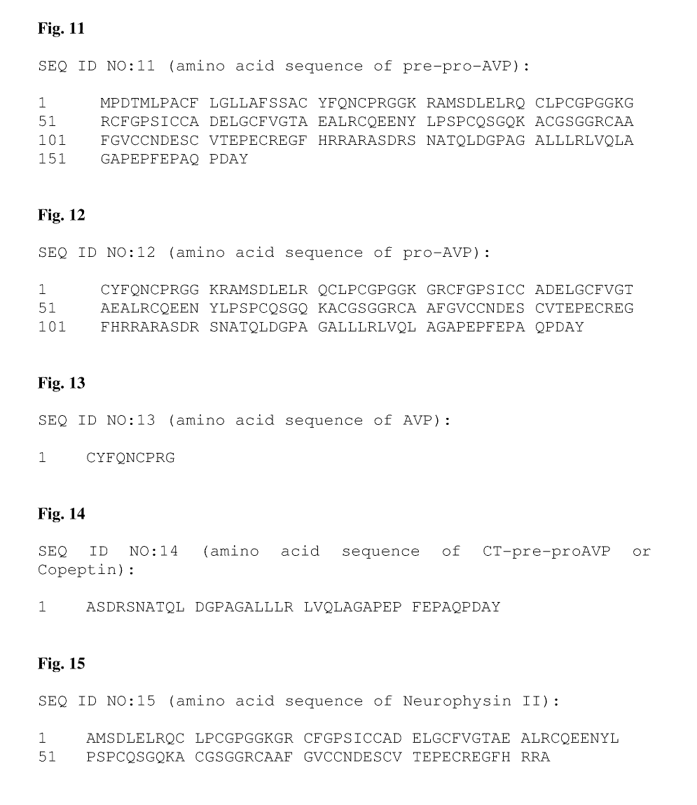

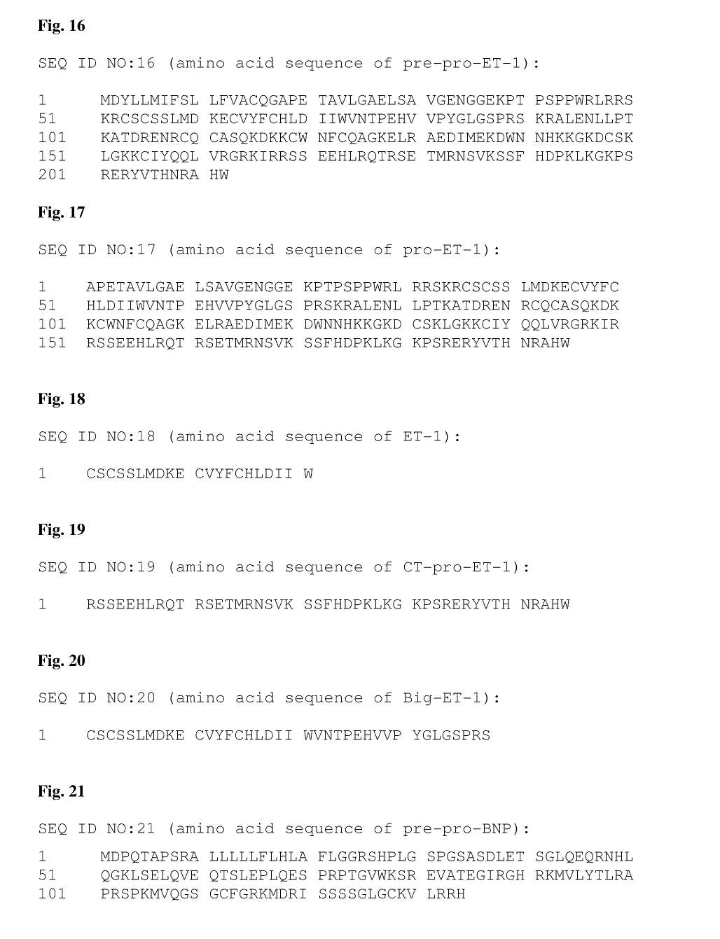

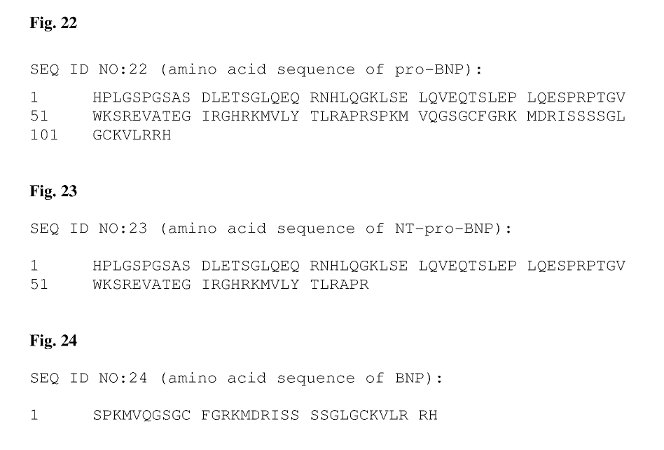

Plasma MR-proANP₅₃₋₉₀ (A) and serum insulin (B) during the oral glucose tolerance test in non obese normotensive subjects (black diamonds), normotensive subjects with central obesity (white diamonds) and hypertensive subjects (black triangles); * p<0.05 non obese vs. obese, normotensive subjects; † p<0.001 obese normotensive vs. hypertensive subjects; ‡ p<0.01 hypertensive vs. non obese normotensive subjects.

(C) Supression of plasma MR-proANP₅₃₋₉₀ levels in the hyperinsulinemic, euglycemic clamps. (D) Delta MR-proANP₅₃₋₉₀ (0-120 min) in subjects with low and high insulin sensitivity, determined as glucose infusion rate (GIR) values in the steady-state of the clamp below the 25^{th} and above the 75^{th} percentile, respectively. Data are shown by box-and-whiskers-plots. The box extends from the 25^{th} to the 75^{th} percentile, with a line at the median indicating the 50^{th} percentile. The whiskers represent the ranges extending from the lowest to the highest value.

FIG. 26

Comparison of relative concentrations of NT-proBNP and MR-proANP₅₃₋₉₀ after oGTT in n=10 subjects.

FIG. 27

(Metabolic syndrome)—correlation of insulin sensitivity with relative MR-proANP change at 120 min [insulin-sensitivity-index (ISI or SD]:—for healthy persons t=0 ISI>4,5 [fasting];—t=30, 60, 90 and 120 min ISI≧6;—for persons with insulin resistance t=30, 60, 90 and 120 min;—ISI<6.

### EXAMPLES

Research Design and Methods

Study Protocol 1 (OGTT and Clamp)

The study protocol was approved by the ethical committees of the Potsdam University and Charite University of Medicine, Berlin, Germany. Before the study, informed written consent was obtained from all participants.

Study Design

The subjects are part of an ongoing case-control association study of the aetiology of the metabolic syndrome and type 2 diabetes mellitus (Metabolic Syndrome Berlin-Potsdam Study, MESY-BEPO). In Potsdam and Berlin, Germany, volunteers from the general population were recruited. The baseline examination included anthropometric measurements, blood sampling, a 75 g oral glucose tolerance test (oGTT) and personal interview on lifestyle habits and medical history. A subgroup of this population (n=31) underwent hyperinsulinemic, euglycemic clamps, which was conducted on a separate day after the oGTT.

Subjects

One hundred and eight non hypertensive subjects (55 non obese and 53 with central obesity) and 54 patients with an essential hypertension were studied. Hypertension was defined as systolic blood pressure ≧140 mm Hg, diastolic blood pressure ≧90 mm Hg, or use of antihypertensive therapy. All drug treated hypertensive subjects had a stable medication in the least six month prior the study. Subjects with elevations in liver enzymes more than twice the respective upper normal limits, or with elevated serum creatinine concentrations (>1.3 mg/dl) or with severe conditions including generalized inflammation, heart failure or end-stage malignant diseases were excluded from the study. All subjects were instructed to maintain their normal physical activity and to consume a normal diet containing 200 g of carbohydrate during three days before oGTT and clamp test. Subjects with antidiabetic therapy or newly diagnosed type 2 diabetes mellitus were excluded from the examination. Definitions of disturbances in the glucose metabolism were based on the 1997 American Diabetes Association criteria for glucose values obtained after an overnight fast and a two-hour 75 g oGTT (2000 Report of the Expert Committee on the Diagnosis and Classification of Diabetes Mellitus. Diabetes Care 23 Suppl 1:S4-19).

Experimental Procedures

All tests were performed in the morning after 12 hours overnight fast. BP was measured by a trained study nurse using an Omron® HEM705CP manometer (Omron, Germany) with patients in the sitting position. Three measurements were taken at 2-min intervals and the average was used to define clinical systolic and diastolic blood pressures. For oGTT venous blood samples were drawn at 0, 30, 60, 90, 120 and 180 min relative to the oral glucose loading. Euglycemic, hyperinsulinemic clamp:

Hyperinsulinemic euglycemic clamps were performed for 120 min using 100 mU of human insulin per m2 of the body surface per min (Actrapid; Novo Nordisk, Bagsvard, Denmark) and a variable infusion of 20% glucose (Serag Wiessner, Naila, Germany) (DeFronzo R A, et al. 1979 Glucose clamp technique: a method for quantifying insulin secretion and resistance. Am J Physiol 237:E214-23). In the steady-state condition of the clamp, capillary blood glucose was adjusted at 5.5 mmol/l for at least 60 min A deviation of a single capillary glucose concentration of >10% during assumed steady-state conditions was defined as non-steady state. Throughout the clamp, capillary blood glucose concentrations were monitored every 5 min and used to regulate plasma glucose by the adjustment of a variable infusion of glucose.

Analytical Procedures

All venous blood samples were immediately centrifuged and frozen at −70° C. until analyzed. Capillary blood glucose concentrations were determined using a glucose oxidase method on Dr. Müller G-L (Dr. Willer Glucose analyzer, Freital, Germany). Serum triglycerides, total cholesterol and HDL-cholesterol were determined by standard enzymatic assays, and LDL-cholesterol calculated from these data (certified laboratory for clinical chemistry). HbA1c was determined using a Hi-Auto A1C HA-8140 system (Menarini Diagnostics, Germany). Serum insulin was measured using a commercial enzyme-linked immunosorbent assay (Insulin ELISA, Mercodia AB, Uppsala, Sweden). Homeostasis Model Assessment Insulin Resistance (HOMA-IR) was calculated as fasting insulin (IU/L)×fasting glucose (mmol/L)/22.5 (Matthews D R, et al. 1985 Homeostasis model assessment: insulin resistance and beta-cell function from fasting plasma glucose and insulin concentrations in man. Diabetologia 28:412-9).

Human plasma MR-proANP53-90 was determined as described previously (Morgenthaler N G, et al. 2004 Immunoluminometric assay for the midregion of pro-atrial natriuretic peptide in human plasma. Clin Chem 50:234-6).

NT-proBNP was determined by an electrochemiluminescence immunoassay (ELICIA, Roche Diagnostics, Basel, Switzerland).

Statistical Analysis

We divided the study population into three groups: patients with essential hypertension (n=54), non hypertensive subjects with central obesity (n=53), and non hypertensive subjects without central obesity (n=55). Central obesity was diagnosed according to the ATP III-defined metabolic syndrome criteria (National Cholesterol Education Program (NCEP) Expert Panel on Detection, Evaluation, and Treatment of High Blood Cholesterol in Adults (Adult Treatment Panel III). Third report of the national Cholesterol Education Program (NCEP) expert panel on detection, evaluation, and treatment of high blood cholesterol in adults (Adult Treatment Panel III). Final report. Circulation 2002; 106:3143-3421).

General characteristics are given as mean±SD. All other data are presented as means±SEM. All data were log-transformed before analysis. Delta MR-proANP53-90 from 0 to 180 min was calculated in oGTT. Group comparison was performed by ANOVA followed by the Sidak test as post hoc multiple group comparison. Repeated measures ANOVA analyses were used for comparison of the time-courses of MR-proANP53-90 during oGTT between three groups. Correlation analysis was made by Pearson's correlation. In clamp experiments, insulin sensitivity was determined as glucose infusion rate (mg per kg body weight·min⁻¹) in the steady-state of the clamp test (least 30 min) divided by circulating insulin concentration in steady-state (pmol/l). The nonparametric Wilcoxon's signed-rank test for paired samples was used to compare data from the baseline and in the steady-state. P values<0.05 were considered significant in all analyses. All statistical analyses were performed using SPSS for Windows 14 (SPSS Inc., Chicago, Ill.).

Results

The characteristics of the study population are summarised in Table 1. Obese normotensive subjects were more insulin resistant compared with non obese normotensive subjects: they had higher fasting insulin and blood glucose concentrations, lower HDL-cholesterol concentration and triglyceride levels, and higher SPB and DBP. Except higher age, HbA1c level and SBP, obese subjects with hypertension were comparable in BMI, waist circumference and insulin resistance with obese normotensive subjects.

The lowest fasting concentrations of MR-proANP53-90 were observed in obese normotensive subjects, compared with non obese normotensive subjects and obese subjects with hypertension (53.9±28.0 pmol/l vs. 64.1±25.6 pmol/l and 77.5±30.8 pmol/l). After adjustment for age and BMI, the differences remained significant. Fasting MR-proANP53-90 levels correlated significantly and positively with age (r=0.429, p<0.0001), HDL-cholesterol (r=0.270, p=0.006), and negatively with BMI (r=−0.313, p=0.001), DBP (r=−0.251, p=0.009), fasting insulin (r=−0.276, p=0.004) and HOMA-IR index (r=−0.268, p=0.005) in normotensive subjects. By contrast, in obese subjects with hypertension positive correlations with MR-proANP53-90 were restricted to age (r=0.546, p<0.0001).

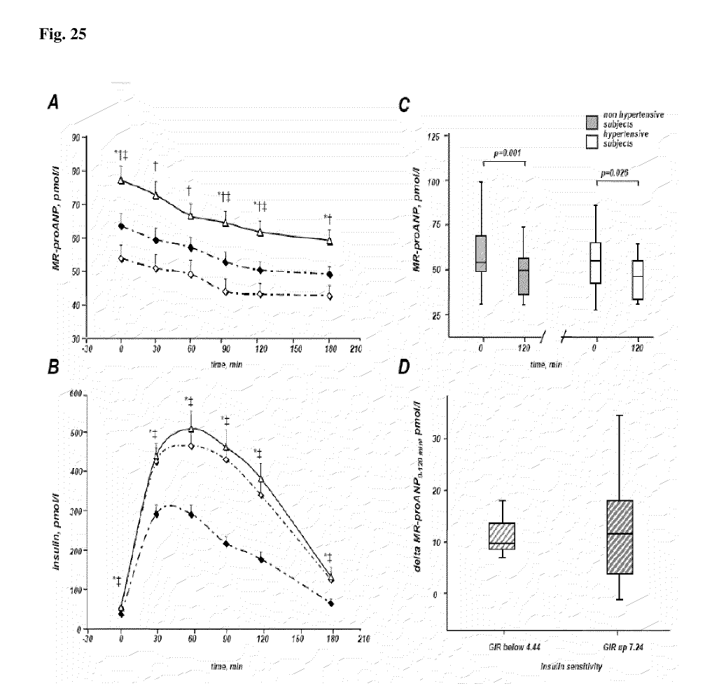

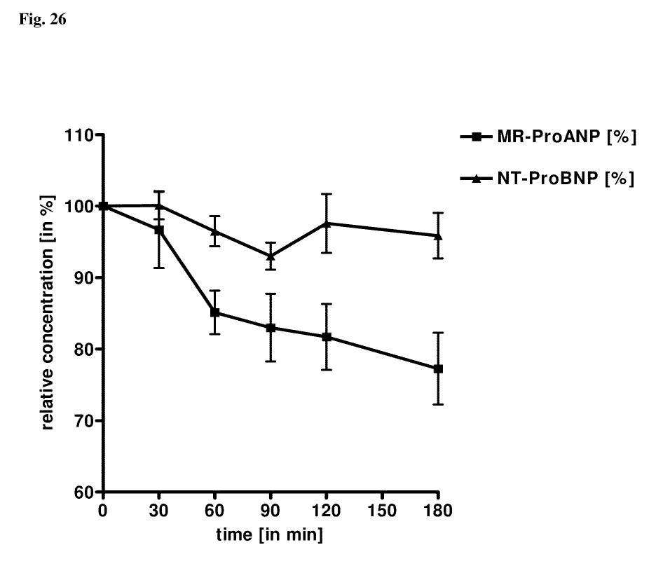

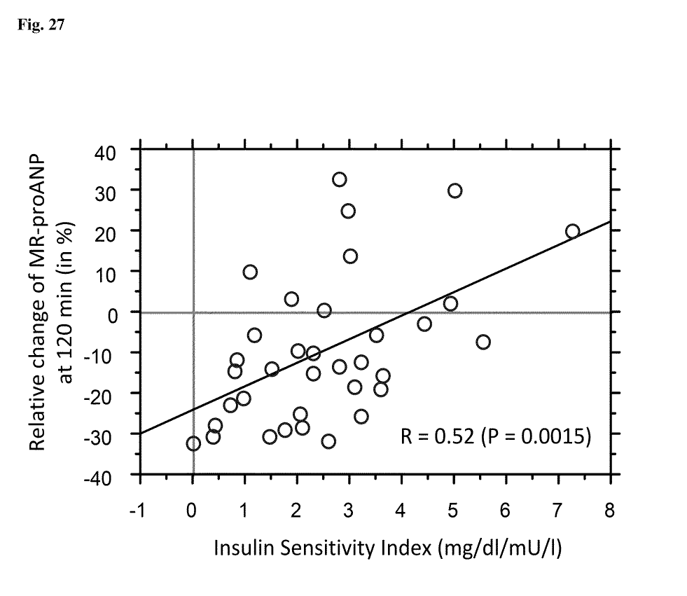

In all study subjects, MR-proANP53-90 levels decreased rapidly at 30 min after oral glucose challenge and remained suppressed during the entire test (p<0.0001; for basal levels vs. levels at 30, 60, 90, 120 and 180 min of the oGTT). Post challenge concentrations of MR-proANP53-90 were significantly lower (FIG. 25-A) from 90 to 180 min in obese normotensive subjects compared with non obese normotensive subjects and obese hypertensive subjects. However, the relative suppression of MR-proANP53-90 at 180 min was similar (20.0±13.4% in non obese normotensive subjects vs. 21.4±19.5% in obese normotensive subjects vs. 21.2±13.4% in hypertensive subjects, NS). Fasting and post glucose challenge levels of insulin were significantly lower in obese normotensive subjects to (FIG. 25B) and correlated negatively with fasting and post challenge levels of MR-proANP53-90 in normotensive subjects from 60 to 180 min (r=−0.198-−0.358; p<0.0001 −0.05), while these correlations were much weaker in hypertensive subjects. There was no correlation of fasting and post challenge blood glucose levels with MR-proANP53-90 levels during oGTT in normotensive and hypertensive subjects (data not shown).

Although BNP appears to play a minor role physiologically in healthy subjects, it is usually regulated in a similar manner to ANP. We therefore tested whether NT-proBNP levels also decline in response to oral glucose challenges. NT-proBNP levels were assessed in 10 subjects and compared to MR-proANP53-90 in this subgroup. Indeed, the relative concentrations of NT-proBNP also declined after the glucose challenge, but the response was more transient and smaller compared to MR-proANP53-90 (FIG. 26).

Thirty-one obese individuals (17 normotensive subjects and 14 hypertensive subjects) underwent euglycemic hyperinsulinemic clamps. Normotensive subjects were matched for age, BMI, waist circumference and insulin sensitivity with hypertensive subjects (mean±SE; 30.6±3.5 kg/m2 vs. 32.0±3.4 kg/m2, p=0.341; 101.0±7.3 cm vs. 103.6±8.8 cm, p=0.526; 6.1±1.6 mg/kg body weight×min-1 vs. 5.5±2.0 mg/kg body weight×min-1, p=0.388; respectively). Fasting blood glucose, insulin and MR-proANP53-90 levels were not different between normotensive and hypertensive subjects (mean±SE; 5.2±0.5 mmol/l vs. 5.4±0.6 mmol/l; p=0.147; 66.0±40.2 pmol/l vs. 76.2±39.0 pmol/l; p=0.470; 59.5±18.3 pmol/l vs. 61.0±31.5 pmol/l; p=0.874; respectively). In the euglycemic clamp, circulating insulin levels increased to 1223 (range 708-2106) pmol/l in normotensive subjects and to 1247 (range 430-1476) pmol/l in hypertensive subjects at 120 min of the clamp (p=0.489). In both groups MR-proANP53-90 levels were significantly decreased at 120 min of the clamp compared with basal values, but they did not differ between both groups (ΔMR-proANP53-90 0-120 min 11.1 (−1.3-25.1) pmol/1 in normotensive subjects vs. 8.5 (−35.2-34.6) pmol/l hypertensive subjects; p=0.297) (FIG. 25-C). No difference was observed in the suppression of MR-proANP53-90 in subjects with low and high insulin sensitivity (determined as glucose infusion rate value below the 25^{th} and above the 75^{th} percentile, respectively) (FIG. 25-D).

Mean MR-ProADM concentration in healthy individuals (n=264) was 0.33 nmol/L (standard deviation 0.07 nmol/L), range 0.1-0.64 nmol/L, 99^{th} percentile was 0.52 nmol/L, 97.5^{th} percentile was 0.49 nmol/L, 2.5^{th} percentile was 0.17 nmol/L, 1^{st} percentile was 0.14 nmol/L. The lower detection limit of the assay was 0.08 nmol/L (Morgenthaler et al. 2005. Clin Chem 51(10):1823-1829).

Median MR-ProANP concentration in healthy individuals (n=325) was 45 pmol/L, range 9.6-313 pmol/L, 99^{th} percentile was 197.5 pmol/L, 97.5^{th} percentile was 163.9 pmol/L, 2.5^{th} percentile was 18.4 pmol/L, 1^{st} percentile was 13.6 pmol/L. The lower detection limit of the assay was 6.0 pmol/L (Morgenthaler et al. 2004. Clin Chem 50(1):234-236).

Median CT-ProAVP concentration in healthy individuals (n=359) was 4.2 pmol/L, range 1-13.8 pmol/L, 99^{th} percentile was 13.5 pmol/L, 97.5^{th} percentile was 11.25 pmol/L, 2.5^{th} percentile was 1.7 pmol/L. The lower detection limit of the assay was 1.7 pmol/L (Morgenthaler et al. 2006. Clin Chem 52(1):112-119). 9 individuals out of 359 had CT-proAVP-values below the lower detection limit and were defined as 1.0 pmol/L.

Mean CT-ProET1 concentration in healthy individuals (n=326) was 44.3 pmol/L (standard deviation 10.6 pmol/L), range 10.5-77.4 pmol/L, 99^{th} percentile was 72.8 pmol/L, 97.5^{th} percentile was 66.6 pmol/L, 2.5^{th} percentile was 24.8 pmol/L, 1^{st} percentile was 17.4 pmol/L. The lower detection limit of the assay was 0.4 pmol/L (Papassotiriou et al. 2006. Clin Chem 52(6):1144-1151).

Mean NT-proBNP concentration in healthy individuals (n=2264) was 5.94 pmol/l (standard deviation 7.36 pmol/l), the median was 3.25 pmol/l, 97.5^{th} percentile was 19.94 pmol/l and 95^{th} percentile was 17.58 pmol/l. The lower detection limit of the assay was 0.59 pmol/l.

TABLE 1
Clinical and biochemical characteristics of the study subjects

| | Non obese, non | Obese, non | Obese, |
|---|---|---|---|
| | hypertensive | hypertensive | hypertensive |
| | subjects | subjects | subjects |
| | (n = 55) | (n = 53) | (n = 54) |
| Clinical characteristics | | | |
| Age (years) | 47.1 ± 13.7 | 47.9 ± 10.5 | 60.1 ± 8.8 ^{a, d} |
| Sex (female; %) | 74.2 | 73.4 | 66.2 |
| BMI (kg/m²) | 23.4 ± 2.3 | 30.0 ± 5.0 ^{a} | 30.0 ± 4.0 ^{a} |
| Waist circumference (cm) | 79.8 ± 8.7 | 100.0 ± 10.4 ^{a} | 100.1 ± 11.4 ^{a} |
| Systolic blood pressure (mm Hg) | 111.3 ± 12.1 | 119.5 ± 13.6 ^{a} | 130.9 ± 16.8 ^{a, d} |
| Diastolic blood pressure (mm Hg) | 70.6 ± 8.2 | 78.5 ± 8.3 ^{a} | 81.6 ± 10.9 ^{a} |
| Biochemical characteristics | | | |
| Fasting blood glucose (mmol/l) | 4.7 ± 0.4 | 5.0 ± 0.5 ^{b} | 5.0 ± 0.6 ^{b} |
| HbA1c (%) | 5.3 ± 0.4 | 5.3 ± 0.4 | 5.6 ± 0.4 ^{a, d} |
| HDL cholesterol (mmol/l) | 1.5 ± 0.3 | 1,.3 ± 0.3 ^{a} | 1.3 ± 0.3 ^{a} |
| Triglycerides (mmol/l) | 1.0 ± 0.6 | 1.6 ± 0.9 ^{b} | 1.6 ± 0.8 ^{a} |
| Fasting insulin (pmol/l) | 36.8 ± 47.0 | 52.9 ± 34.9 ^{a} | 54.1 ± 35.8 ^{a} |
| HOMA_{IR} (mU mmol/l) | 1.3 ± 1.9 | 2.0 ± 1.4 ^{a} | 2.0 ± 1.5 ^{a} |
| proANP (pmol/l) 0 min | 64.1 ± 25.6 | 53.9 ± 28.0 ^{c} | 77.5 ± 30.8 ^{d} |
| delta proANP_{(0-180 min)} (pmol/l) | 14.7 ± 11.9 | 10.9 ± 11.0 ^{c} | 18.2 ± 15.3 ^{d} |

Unless otherwise indicated, values are means ± SD. All values are unadjusted.
^{a} p < 0.0001;
^{b} p < 0.01;
^{c} p < 0.05 vs. non obese, non hypertensive subjects and
^{d} p < 0.0001;
^{e} p < 0.05 vs. obese non hypertensive subjects. Obesity was defined as “central obesity” by ATIII Criteria for metabolic syndrome: waist circumference for women >88 cm and >102 cm for men.

The study protocol was approved by the ethical committee of the Charite University of Medicine, Berlin, Germany. Before the study, informed written consent was obtained from all participants.

Analytical Procedures (bitte überprüfen and überarbeiten/ergänzen)

All venous blood samples were immediately centrifuged and frozen at −70° C. until analyzed. Capillary blood glucose concentrations were determined using a glucose oxidase method on Dr. Müller G-L (Dr. Willer Glucose analyzer, Freital, Germany). HbA1c was determined using a Hi-Auto A1C HA-8140 system (Menarini Diagnostics, Germany). Serum insulin was measured using a commercial enzyme-linked immunosorbent assay (Insulin ELISA, Mercodia AB, Uppsala, Sweden). Homeostasis Model Assessment Insulin Resistance (HOMA-IR) was calculated as fasting insulin (IU/L)×fasting glucose (mmol/L)/22.5 (Matthews D R, et al. 1985 Homeostasis model assessment: insulin resistance and beta-cell function from fasting plasma glucose and insulin concentrations in man. Diabetologia 28:412-9).

Human plasma MR-proANP53-90 was determined as described previously (Morgenthaler N G, et al. 2004 Immunoluminometric assay for the midregion of pro-atrial natriuretic peptide in human plasma. Clin Chem 50:234-6).

NT-proBNP was determined by an electrochemiluminescence immunoassay (ELICIA, Roche Diagnostics, Basel, Switzerland).

1. An in vitro-method for diagnosis, prognosis and/or monitoring/therapy follow-up of a disorder of the metabolic system and/or cardiovascular system and/or insulin resistance in a subject, comprising:
a) providing a sample from said subject,
b) determining the relative level of one or more cardiovascular markers in said sample,
c) using the relative level of said one or more cardiovascular peptides for the diagnosis, prognosis and/or monitoring/therapy follow-up of a disorder of the metabolic system and/or cardiovascular system and/or insulin resistance in said subject.

2. The in vitro-method according to claim 1, wherein the postprandial relative level of one or more cardiovascular markers is determined

3. The in vitro-method according to claim 1, wherein the cardiovascular marker is selected from the group comprising ANP, BNP, ET-1, ADM, AVP and fragments thereof and pro-hormones and fragments thereof.

4. The in vitro-method according to claim 1, wherein the relative level of one or more cardiovascular markers is determined with an assay having a sensitivity 1 nmol/L or lower, preferably 100 pmol/L or lower, more preferably 10 pmol/l or lower, even more preferably 1 pmol/L or lower, most preferably 0.5 pmol/l or lower.

5. The in vitro-method according to claim 1, wherein determining the relative level comprises the steps of:
a. determining the basal level of one or more cardiovascular markers in said subject,
b. determining the postprandial level of said one or more cardiovascular markers,
c. calculating the relative level of one or more cardiovascular markers from the values obtained in steps a and b.

6. The in vitro-method according to claim 1, wherein level of said cardiovascular markers in said subject is determined with an immunoassay.

7. The in vitro-method according to claim 1, wherein one or more of the cardiovascular markers is selected from the group comprising proANP or fragments thereof, pro-ET-1 or fragments thereof, pro-BNP or fragments thereof, pro-AVP or fragments thereof, pro-ADM or fragments thereof.

8. A method for determining the change of the level of one or more cardiovascular markers of a subject relative to the basal level of said markers of said subject, comprising performing an assay is capable of detecting a decrease of the level of said one or more cardiovascular markers and capable of detecting an increase of the level of said one or more cardiovascular markers.

9. The method according to claim 8, wherein the change is an increase or a decrease, and wherein the assay has sensitivity of 1 nmol/L or lower.

10. The method according to claim 8 for determining the postprandial change of the level of one or more cardiovascular markers of a subject relative to the basal level of said markers of said subject.

11. The method according to claim 7, wherein the assay is an immunoassay.

12. The method according to claim 7 for diagnosis, prognosis and/or monitoring/therapy follow-up of a disorder of the metabolic system and/or cardiovascular system and/or insulin resistance in a subject.

13. The in vitro-method according to claim 1, wherein additionally the level of one or more further markers or clinical parameters having predictive value for classifying the propensity of said patient for a disorder of the metabolic system and/or cardiovascular system is determined, wherein the clinical parameters may be any parameter which might influence said propensity, such as for instance age, gender, prior history of diseases, in particular hypertension, obesity, in particular central obesity, body mass index, genetic predisposition/family history, ethnic background, patient's habits which affect said propensity, such as smoking, alcohol consumption, diet, exercise or medication.

14. The use of a cardiovascular peptide for diagnosis, prognosis and/or monitoring/therapy follow-up of a disorder of the metabolic system and/or cardiovascular system of a patient.


SEQUENCE LISTING (24 sequences)

SEQ ID NO: 1 - protein, 185 aa, Homo sapiens
MKLVSVALMYLGSLAFLGADTARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 2 - protein, 164 aa, Homo sapiens
ARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 3 - protein, 20 aa, Homo sapiens
ARLDVASEFRKKWNKWALSR

SEQ ID NO: 4 - protein, 40 aa, Homo sapiens
ELRMSSSYPTGLADVKAGPAQTLIRPQDMKGASRSPEDSS

SEQ ID NO: 5 - protein, 52 aa, Homo sapiens
YRQSMNNFQGLRSFGCRFGTCTVQKLAHQIYQFTDKDKDNVAPRSKISPQGY

SEQ ID NO: 6 - protein, 153 aa, Homo sapiens [sequence omitted]

SEQ ID NO: 7 - protein, 126 aa, Homo sapiens
NPMYNAVSNADLMDFKNLLDHLEEKMPLEDEVVPPQVLSEPNEEAGAALSPLPEVPPWTGEVSPAQRDGGALGRGPWDSSDRSALLKSKLRALLTAPRSLRRSSCFGGRMDRIGAQSGLGCNSFRY

SEQ ID NO: 8 - protein, 28 aa, Homo sapiens
SLRRSSCFGGRMDRIGAQSGLGCNSFRY

SEQ ID NO: 9 - protein, 98 aa, Homo sapiens
NPMYNAVSNADLMDFKNLLDHLEEKMPLEDEVVPPQVLSEPNEEAGAALSPLPEVPPWTGEVSPAQRDGGALGRGPWDSSDRSALLKSKLRALLTAPR

SEQ ID NO: 10 - protein, 38 aa, Homo sapiens
PEVPPWTGEVSPAQRDGGALGRGPWDSSDRSALLKSKL

SEQ ID NO: 11 - protein, 164 aa, Homo sapiens
MPDTMLPACFLGLLAFSSACYFQNCPRGGKRAMSDLELRQCLPCGPGGKGRCFGPSICCADELGCFVGTAEALRCQEENYLPSPCQSGQKACGSGGRCAAFGVCCNDESCVTEPECREGFHRRARASDRSNATQLDGPAGALLLRLVQLAGAPEPFEPAQPDAY

SEQ ID NO: 12 - protein, 145 aa, Homo sapiens
CYFQNCPRGGKRAMSDLELRQCLPCGPGGKGRCFGPSICCADELGCFVGTAEALRCQEENYLPSPCQSGQKACGSGGRCAAFGVCCNDESCVTEPECREGFHRRARASDRSNATQLDGPAGALLLRLVQLAGAPEPFEPAQPDAY

SEQ ID NO: 13 - protein, 9 aa, Homo sapiens
CYFQNCPRG

SEQ ID NO: 14 - protein, 39 aa, Homo sapiens
ASDRSNATQLDGPAGALLLRLVQLAGAPEPFEPAQPDAY

SEQ ID NO: 15 - protein, 93 aa, Homo sapiens
AMSDLELRQCLPCGPGGKGRCFGPSICCADELGCFVGTAEALRCQEENYLPSPCQSGQKACGSGGRCAAFGVCCNDESCVTEPECREGFHRRA

SEQ ID NO: 16 - protein, 212 aa, Homo sapiens
MDYLLMIFSLLFVACQGAPETAVLGAELSAVGENGGEKPTPSPPWRLRRSKRCSCSSLMDKECVYFCHLDIIWVNTPEHVVPYGLGSPRSKRALENLLPTKATDRENRCQCASQKDKKCWNFCQAGKELRAEDIMEKDWNNHKKGKDCSKLGKKCIYQQLVRGRKIRRSSEEHLRQTRSETMRNSVKSSFHDPKLKGKPSRERYVTHNRAHW

SEQ ID NO: 17 - protein, 195 aa, Homo sapiens
APETAVLGAELSAVGENGGEKPTPSPPWRLRRSKRCSCSSLMDKECVYFCHLDIIWVNTPEHVVPYGLGSPRSKRALENLLPTKATDRENRCQCASQKDKKCWNFCQAGKELRAEDIMEKDWNNHKKGKDCSKLGKKCIYQQLVRGRKIRRSSEEHLRQTRSETMRNSVKSSFHDPKLKGKPSRERYVTHNRAHW

SEQ ID NO: 18 - protein, 21 aa, Homo sapiens
CSCSSLMDKECVYFCHLDIIW

SEQ ID NO: 19 - protein, 45 aa, Homo sapiens
RSSEEHLRQTRSETMRNSVKSSFHDPKLKGKPSRERYVTHNRAHW

SEQ ID NO: 20 - protein, 38 aa, Homo sapiens
CSCSSLMDKECVYFCHLDIIWVNTPEHVVPYGLGSPRS

SEQ ID NO: 21 - protein, 134 aa, Homo sapiens
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 22 - protein, 108 aa, Homo sapiens
HPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 23 - protein, 76 aa, Homo sapiens
HPLGSPGSASDLETSGLQEQRNHLQGKLSELQVEQTSLEPLQESPRPTGVWKSREVATEGIRGHRKMVLYTLRAPR

SEQ ID NO: 24 - protein, 32 aa, Homo sapiens
SPKMVQGSGCFGRKMDRISSSSGLGCKVLRRH